(19) United States
(12) Patent Application Publication
(10) Pub. No.: US 2018/0028429 A1
(43) Pub. Date: Feb. 1, 2018

(54) MIXTURE OF ACTIVE INGREDIENTS FOR HAIRSTYLING COMPOSITIONS

(71) Applicant: Henkel AG & Co. KGaA, Duesseldorf (DE)

(72) Inventors: Thorsten Knappe, Schenefeld (DE); Justine Caspar, Biswihr (FR)

(73) Assignee: Henkel AG & Co. KGaA, Duesseldorf (DE)

(21) Appl. No.: 15/659,638

(22) Filed: Jul. 26, 2017

(30) Foreign Application Priority Data
Jul. 26, 2016 (DE) 10 2016 213 643.7
Nov. 8, 2016 (DE) 10 2016 221 830.1

(51) Int. Cl. A61K 8/81; A61Q 5/06; A61K 8/92; A61K 8/73; A61K 8/36

(52) U.S. Cl. CPC A61K 8/817; A61K 8/736; A61K 8/361; A61K 8/922; A61Q 5/06; A61K 2800/30; A61K 2800/596

(57) ABSTRACT

A cosmetic composition for the temporary shaping of keratin-containing fibers is provided herein. The cosmetic composition includes in a cosmetic carrier, based upon the total weight of the cosmetic composition, from about 0.05 to about 2.0% by weight of chitosan or a chitosan derivative, from about 0.05 to about 2.0% by weight of a C₁-C₄ carboxylic acid, and from about 0.05 to about 4.0% by weight of a vinylpyrrolidone homopolymer. The cosmetic composition is free of vinyl acetate/vinylpyrrolidone copolymer and cationic cellulose compounds, which result from the reaction of hydroxyethylcellulose with a dimethyldiallylammonium reactant, or is free of vinyl acetate/vinylpyrrolidone copolymer and N-methylvinylimidazole/vinylpyrrolidone copolymer.

### CROSS-REFERENCE TO RELATED APPLICATION

This application claims priority to German Patent Application No. 10 2016 213 643.7, filed Jul. 26, 2016 and German Patent Application No. 10 2016 221 830.1, filed Nov. 8, 2016, which are incorporated herein by reference in their entirety.

### TECHNICAL FIELD

The present disclosure relates to cosmetic agents for temporarily shaping keratin-containing fibers, which contain, in a cosmetic carrier, a combination of at least one chitosan compound, a C₁-C₄ carboxylic acid and a polyvinylpyrrolidone homopolymer, but are free of a vinyl acetate/vinylpyrrolidone copolymer and cationic cellulose compounds, which result from the reaction of hydroxyethylcellulose with a dimethyldiallylammonium reactant, in particular dimethyldiallylammonium chloride, or are free of vinyl acetate/vinylpyrrolidone copolymer and N-methylvinylimidazole/vinylpyrrolidone copolymer.

### BACKGROUND

The invention furthermore relates to methods for temporarily shaping keratin-containing fibers as well as to the use of the cosmetic composition for improving the curl retention, especially the high humidity curl retention.

Agents for shaping temporarily are known in principle. They usually contain synthetic polymers as shaping components. Preparations, which contain a dissolved or dispersed polymer, can be applied on the hair by employing propellants or by a pump mechanism. However, hair gels and hair waxes are usually not applied directly on the hair and, instead, are distributed by employing a comb or the hands.

The most important property of an agent for the temporary deformation of keratin-containing fibers, also referred to as styling composition in the following, is to ensure that the treated fibers are held strongly in the form produced. If the keratin-containing fibers are human hair, it is customary to speak also of a long-lasting styling or of a high degree of holding of the styling composition.

As a rule, cosmetic styling compositions contain individual polymers specifically tailored to achieve a very specific effect. If different effects are to be achieved, it is necessary to add several polymers. However, if too many different polymers are used, this may entail a number of disadvantages. Thus, problems may arise in the formulation, for example, because the polymers react with one another or with other components of the composition and precipitation or decomposition occurs. Certain polymers also tend to deposit so firmly on the skin and, particularly, on the hair so stably, that they are no longer removed completely during ordinary washing, resulting in an undesirable accumulation of the polymer and ultimately become a burden on the skin or hair.

There is therefore a constant need for polymers or suitable combinations of fewer polymers, which simultaneously have as many of the desired properties as possible.

For example, in the case of hair styling compositions, it is necessary that the polymers used hold the treated hair as strongly as possible. In addition to holding the hair strongly, hair styling compositions must fulfill a whole series of further requirements. These may be divided roughly into properties of the hair, properties of the particular formulation, for example, properties of the foam, the gel or the sprayed aerosol, and properties relating to the handling of the hair styling composition, with special importance being attached to the properties of the hair. Particular mention should be made of moisture resistance, low stickiness and a balanced conditioning effect. Furthermore, a hairstyling composition should be usable universally for all types of hair if possible.

How the hairstyle holds up in general and, with curled hair, the curl retention specifically are special requirements for styling compositions. The so-called curl retention is also a measure of how the curls hold up. Usually the curl retention is worse when the treated hair is in a damp environment, since the tendency of the hair to absorb moisture, that is, water, decreases the curl retention.

### BRIEF SUMMARY

A cosmetic composition for the temporary shaping of keratin-containing fibers is provided herein. The cosmetic composition includes in a cosmetic carrier, based upon the total weight of the cosmetic composition, from about 0.05 to about 2.0% by weight of chitosan or a chitosan derivative, from about 0.05 to about 2.0% by weight of a C₁-C₄ carboxylic acid, and from about 0.05 to about 4.0% by weight of a vinylpyrrolidone homopolymer. The cosmetic composition is free of vinyl acetate/vinylpyrrolidone copolymer and cationic cellulose compounds, which result from the reaction of hydroxyethylcellulose with a dimethyldiallylammonium reactant, or is free of vinyl acetate/vinylpyrrolidone copolymer and N-methylvinylimidazole/vinylpyrrolidone copolymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was therefore an object of the present disclosure to provide compositions for the temporary deformation of keratin-containing fibers which bring about a markedly improved curl retention, in particular, a markedly improved high humidity curl retention, that is, a better curl retention level even in a humid environment, preferably with a further improvement in the retention of the styling of the hair and in the feel of the hair (soft feel and elasticity).

A further object of the present disclosure was to increase and/or maintain the hair fullness, especially in a humid environment.

This objective was accomplished by a special combination of active ingredients, comprising a chitosan, a C₁-C₄ carboxylic acid and a vinylpyrrolidone homopolymer.

Mousses, based on chitosan and polyvinylpyrrolidone, are described, for example, in the German patent application DE 195 31 145 A1.

Embodiments of the materials and methods and some of their preferred embodiments are exemplified by the following points:
1. A cosmetic material for the temporary shaping of keratin-containing fibers, especially of human hair, containing in a cosmetic carrier, based upon the total weight of the latter:
a) from about 0.05 to about 2.0% by weight of chitosan or a chitosan derivative
b) from about 0.05 to about 2.0% by weight of a C₁-C₄ carboxylic acid
c) from about 0.05 to about 4% by weight of a vinylpyrrolidone homopolymer exemplified in that the cosmetic composition

is free of vinyl acetate/vinylpyrrolidone copolymer and cationic cellulose compounds, which result from the reaction of hydroxyethylcellulose with a dimethyldiallylammonium reactant, especially with dimethyldiallylammonium chloride or

is free of vinyl acetate/vinylpyrrolidone copolymer and N-methylvinylimidazole/vinylpyrrolidone copolymer.

2. A cosmetic material for the temporary shaping of keratin-containing fibers, especially of human hair, containing in a cosmetic carrier, based upon the total weight of the latter:
a) from about 0.05 to about 2.0% by weight of chitosan or a chitosan derivative
b) from about 0.05 to about 2.0% by weight of a C₁-C₄ carboxylic acid
c) from about 0.05 to about 4.0% by weight of a vinylpyrrolidone homopolymer exemplified in that the cosmetic composition is free of vinyl acetate/vinylpyrrolidone copolymer and cationic cellulose compounds, which result from the reaction of hydroxyethylcellulose with a dimethyldiallylammonium reactant, especially with dimethyldiallylammonium chloride.
3. The cosmetic material for the temporary shaping of keratin-containing fibers, especially of human hair, containing in a cosmetic carrier, based upon the total weight of the latter:
a) from about 0.05 to about 2.0% by weight of chitosan or a chitosan derivative
b) from about 0.05 to about 2.0% by weight of a C₁-C₄ carboxylic acid
c) from about 0.05 to about 4.0% by weight of a vinylpyrrolidone homopolymer exemplified in that the cosmetic composition is free of vinyl acetate/vinylpyrrolidone copolymer and N-methylvinylimidazole/vinylpyrrolidone copolymer.
4. The cosmetic composition of one of the preceding claims, exemplified in that the percentage by weight of chitosan or chitosan derivative, preferably of chitosan, in the total weight of the cosmetic composition, is from about 0.1 to about 1.5% by weight and preferably from about 0.2 to about 1.0% by weight.
5. The cosmetic composition of one of the preceding points, exemplified in that the percentage by weight of the C₁-C₄ carboxylic acid in the total weight of the cosmetic composition, is from about 0.1 to about 1.5% by weight and preferably from about 0.2 to about 1.0% by weight.
6. The cosmetic composition of one of the preceding points, exemplified in that the C₁-C₄ carboxylic acid is selected from the group comprising formic acid and lactic acid, preferably lactic acid.
7. The cosmetic composition of one of the preceding points, exemplified in that the percentage by weight of the vinylpyrrolidone homopolymer in the total weight of the cosmetic composition, is from about 0.3 to about 3.0% by weight and preferably from about 0.5 to about 2.5% by weight.
8. The cosmetic composition of one of the preceding points, containing in a cosmetic composition, based on the total weight of the latter:
a) from about 0.05 to about 2.0% by weight of chitosan
b) from about 0.05 to about 2.0% by weight of lactic acid
c) from about 0.05 to about 4.0% by weight of a vinylpyrrolidone homopolymer exemplified in that the cosmetic composition

is free of vinylpyrrolidone/vinyl acetate copolymer and cationic cellulose compounds, which result from the reaction of hydroxyethylcellulose with a dimethyldiallylammonium reactant, especially with dimethyldiallylammonium chloride or

is free of vinyl acetate/vinylpyrrolidone copolymer and N-methylvinylimidazole/vinylpyrrolidone copolymer.

9. The cosmetic composition of one of the preceding points, exemplified in that the cosmetic composition is free cationic cellulose compounds, which result from the reaction of hydroxyethylcellulose with a dimethyldiallylammonium reactant, especially with dimethyldiallylammonium chloride and has the INCI name of polyquaternium-4.
10. The cosmetic composition of one of the preceding points, exemplified in that the cosmetic composition is free of cationic cellulose compounds.
11. The cosmetic composition of one of the preceding points, exemplified in that the composition, based on the total weight thereof, furthermore contains
d) from about 0.01 to about 2.0% by weight, preferably of from about 0.05 to about 1.0% by weight and especially of 0.1 to 0.8% by weight of silicone, preferably of an amino functional silicone.
12. The cosmetic composition of one of the preceding points, exemplified in that the composition, based on the total weight thereof, furthermore contains
e) from about 0.05 to about 2.0% by weight, preferably from about 0.1 to about 1.0% by weight, and in particular from about 0.1 to about 0.5% by weight of at least one nonionic surfactant from the group comprising the addition products of from about 30 to about 60 moles of ethylene oxide and castor oil and hydrogenated castor oil, preferably at least one nonionic surfactant from the group comprising compounds with the INCI names Steareth-30, Ceteareth-30, Oleth-30, Ceteareth-50, PEG-40 hydrogenated castor oil and PEG-60 hydrogenated castor oil, especially PEG-40 hydrogenated castor oil.
13. The cosmetic composition of one of the preceding points, exemplified in that the composition, based on the total weight thereof, furthermore contains
f) from about 0.05 to about 5.0% by weight, especially from about 0.1 to about 4.0% by weight and particularly from about 0.2 to about 3.0% by weight of at least one cationic surfactant.
14. The cosmetic composition of one of the preceding points, exemplified in that the cosmetic composition, based on the total weight thereof, contains from about 50 to about 95%, especially from about 60 to about 92.5 percent and particularly from about 70 to about 90% of water or of a mixture of water and alcohol.
15. The cosmetic composition of one of the preceding points, exemplified in that the cosmetic composition, based on the total weight thereof, contains from about 1 to about 15%, especially from about 2 to about 12.5% and particularly from about 3 to about 10% of a propellant, preferably of propane and/or butane.
16. The cosmetic composition of one of the preceding points, exemplified in that the cosmetic composition is free of hydrophobic, pyrogenic silica.
17. The use of a cosmetic composition of one of the preceding points for the temporary shaping of keratinic fibers.
18. The use of a cosmetic composition of one of the preceding points for improving the curl retention, especially the high moisture curl retention.
19. A method for the temporary shaping of keratinic fibers, especially of human hair, for which the cosmetic composition of one of the preceding points is applied on keratinic fibers.

As contemplated herein, keratin-containing fibers are understood to be wool, fur, feathers and especially human hair.

A first essential component of the cosmetic composition is the chitosan or chitosan derivative.

Chitosans a) are biopolymers and belong to the group of hydrocolloids. Chemically considered, they are partially deacetylated chitins of different molecular weight. For producing chitosans, one starts from chitin, preferably the shell residues of crustaceans, which are available as cheap raw materials in large amounts. Initially, the chitin usually is deproteinized by the addition of bases, demineralized by addition of mineral acids and finally deacetylated by addition of strong bases, the molecular weights being distributed over a broad spectrum. Preferably, those types are used, which have an average molecular weight (weight average) of from about 800,000 to about 1,200,000 dalton, a Brookfield viscosity (1% by weight in glycolic acid) below 5000 mPas, a degree of deacetylation ranging from about 80 to about 88%, and an ash content of less than 0.3% by weight. Aside from chitosans as typical biopolymers, cationically derivatized chitosans (such as, for example, quaternization products) or alkoxylated chitosans as derivatives of chitosan also come into consideration for the purposes as contemplated herein.

Suitable chitosans are freely available commercially, for example, under the trade names Hydagen® CMF (1% by weight of active substance in an aqueous solution with 0,4% by weight of glycolic acid, molecular weight of from about 500,000 to about 5,000,000 g/mole, Cognis), Hydamer® HCMF (chitosan (80% deacetylated), molecular weight from about 50,000 to about 1,000,000 g/mole, Chitinor, formerly Cognis), Kytamer® PC (about 80% by weight of active substance of chitosan pyrrolidone carboxylate (INCI name: Chitosan PCA), Amerchol), Chitolam® NB/101 and Chitosan 90/100/A1® (Chitosan (about 90% deacetylated), BioLog Heppe).

In preferred inventive compositions and based on the total weight of these, the at least one chitosan or the derivatives thereof a), is contained in a total amount of from about 0.1 to about 1.5% by weight and preferably of from about 0.2 to about 1.0% by weight. The use of chitosan is particularly preferred.

The second essential component of embodiments of the cosmetic composition is the C₁-C₄ carboxylic acid, the proportion by weight in the total cosmetic composition preferably being from about 0.1 to about 1.5% by weight and particularly of from about 0.2 to about 1.0% by weight.

Preferred C₁-C₄ carboxylic acids are selected from the group comprising formic acid and lactic acid, lactic acid being particularly preferred.

The third essential component of embodiments of the composition is the vinylpyrrolidone homopolymer. For the cosmetic effect of the compositions, a proportion of from about 0.3 to about 3.0% by weight or of from about 0.5 to about 2.5% by weight of the vinylpyrrolidone homopolymer to the total weight of the cosmetic composition has proven to be advantageous. Vinylpyrrolidone homopolymers (INCI name: PVP) are marketed, for example, by BASF SE under the trade names Luviskol® K 90 and Luviskol® K 85.

Surprisingly, it was possible to increase the cosmetic effect of embodiments of the compositions, especially the time period, for which the hairstyle lasts, further by the exclusion of

vinylpyrrolidone/vinyl acetate copolymer and cationic cellulose compounds, which result from the reaction of hydroxyethylcellulose with dimethyldiallylammonium reactants, especially with dimethyldiallylammonium chloride or

vinyl acetate/vinylpyrrolidone copolymer and N-methylvinylimidazole/vinylpyrrolidone copolymer.

The group of vinyl acetate/vinylpyrrolidone copolymers (INCI name: VP/VA copolymer) includes, for example, the commercial products Luviskol® VA 64 and Luviskol® VA 73 of BASF SE.

N-methylvinylimidazole/vinylpyrrolidone copolymers (INCI name: Polyquaternium-16) are also marketed by BASF under the trade names Luviquat® Style, Luviquat® FC 370, Luviquat® FC 550, Luviquat® FC 905 and Luviquat® HM 552.

Cosmetic composition of a first embodiment, preferred as contemplated herein, are exemplified in that the cosmetic composition is free cationic cellulose compounds, which result from the reaction of hydroxyethylcellulose with a dimethyldiallylammonium reactant, especially with dimethyldiallylammonium chloride and has the INCI name of Polyquaternium-4.

Cationic cellulose derivatives, in particular those, which result from a reaction of hydroxyethylcellulose with a dimethyldiallylammonium reactant (especially with dimethyldiallylammonium chloride) optionally in the presence of further reactants and which carry the INCI name Polyquaternium-4, are sold, for example, under the names Celquat® H 100 and Celquat® L 200 by the National Starch company.

It is particularly preferred if the cosmetic composition is free of cationic cellulose compounds.

To summarize, cosmetic compositions are preferred, which contain, in a cosmetic carrier,

a) from about 0.05 to about 2.0% by weight of chitosan
b) from about 0.05 to about 2.0% by weight of lactic acid and
c) from about 0.05 to about 4.0% by weight of a vinylpyrrolidone homopolymer
and

are free of vinylpyrrolidone/vinyl acetate copolymer and cationic cellulose compounds, which result from the reaction of hydroxyethylcellulose with a dimethyldiallylammonium reactant, especially with dimethyldiallylammonium chloride or

are free of vinyl acetate/vinylpyrrolidone copolymer and N-methylvinylimidazole/vinylpyrrolidone copolymer.

Aside from the essential components, described above, preferred cosmetic compositions contain further optional active or auxiliary materials. The group of these active and auxiliary materials contains, for instance, silicones and surfactants

A first group of preferred care components is formed by the silicones, especially by

alkoxylated dimethicones, preferably from the group of ethoxylated/propoxylated dimethicones;

aminofunctional silicones.

The group of alkoxylated dimethicones includes, for example,

ethoxylated dimethicones with the INCI name of PEG-x dimethicone with x=2 to 20, preferably from about 3 to about 17 and in particular 11 or 12;

the ethoxylated dimethicones with the INCI name of PEG-x dimethicones with x=3 to 25 and preferably from about 4 to about 20;

the ethoxylated/propoxylated dimethicones with the INCI name of PEG/PPG a/b dimethicones, wherein a and b, independently of one another, represent the numbers from about 2 to about 30, preferably from about 12 to about 24 and particularly from about 14 to about 20;

the ethoxylated/propoxylated dimethicones with the INCI name of PEG/PPG c/d dimethicones, wherein c and d, independently of one another, represent the numbers from about 10 to about 25, preferably from about 14 to about 20 and particularly from about 14 to about 16;

the ethoxylated/propoxylated dimethicones with the INCI name of to-PEG/PPG e/f PEG/PPG g/h dimethicones, wherein e, f, g and h, independently of one another, represent the numbers from about 10 to about 20, preferably from about 14 to about 28 and particularly 16.

The group of amino-functional silicones, which have at least one amino group that optionally is substituted, are referred to as amodimethicones according to the INCI declaration.

Preferred cosmetic preparations contain one or more amino-functional silicones of the formula

M(RₐQ_{b}SiO_{(4-a-b)/2})ₓ(R_{c}SiO_{(4-c)/2})_{y}M (Si-1),

R is a hydrocarbon or a hydrocarbon group with 1 to about 6 carbon atoms, preferably methyl,
Q a polar group of the general formula —R¹HZ, preferably a group of the Formula CH₂CH₂CH₂NHCH₂CH₂NH₂,

wherein

R1 represents a divalent connecting group, which is linked to hydrogen and the Z group and composed of carbon and hydrogen atoms, carbon, hydrogen and oxygen atoms or carbon hydrogen and nitrogen atoms, and
Z is an organic, amino functional group, which contains at least one amino functional group, which preferably is a —NHCH₂CH₂NH₂— group or a group of formula CH₂CH₂CH₂NHCH₂CH₂NH₂;
a has values ranging from about 0 to about 2,
b has values ranging from about 1 to about 3,
a+b is less than or equal to 3 and
c) is a number ranging from about 1 to about 3, and
x is a number ranging from about 1 to about 2,000, preferably from about 3 to about 50 and particularly for about 3 to about 25, and
y is a number ranging from about 20 to about 10,000, preferably from about 125 to about 10,000 and particularly from about 150 to about 1,000, and
M is a suitable silicone end group, known in the prior art and preferably trimethylsiloxy.

Examples of R¹ include methylene, ethylene, propylene, hexamethylene, decamethylene CH₂CH(CH₃)CH₂—, phenylene, naphthalene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Preferred, inventive cosmetic preparations contain an aminofunctional silicone of formula

R′ₐG₃₋ₐ-Si(OSiG₂)ₙ-(OSiG_{b}R′_{2-b})ₘ—O-SiG₃₋ₐ-R′ₐ (Si-2),

in which
G Is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between about 0 and about 3, especially 0;
b is a number between about 0 and about 1, especially 1;
m and n are numbers, the sum of which (m+n) is between about 1 and about 2000, preferably between about 50 and about 150, n preferably having values from about 0 to about 1999 and especially from about 49 to about 149 and m preferably has values from about 1 to about 2000 and especially from about 1 to about 10,
R is a monovalent group selected from
   -Q-N(R″)—CH₂—CH₂—N(R″)₂
   -Q-N(R″)₂
   -Q-N⁺(R″)₃A⁻
   -Q-N⁺H(R″)₂
   -Q-N⁺H₂(R″)A⁻
      -Q-N(R″)—CH₂—CH₂—N⁺R″H₂A⁻,
      wherein each Q represents a chemical bond —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ represents identical or different groups from the group comprising —H, -phenyl, -benzyl, —CH₂—CH(CH₃)Ph, the C₁₋₂₀ alkyl groups, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃ or —C(CH₃)₃ and A represents an anion, which preferably is selected from chloride, bromide, iodide or methosulfate.

Particularly preferred cosmetic preparations are exemplified owing to the fact that they contain at least one aminofunctional silicone of formula (Si1-a)

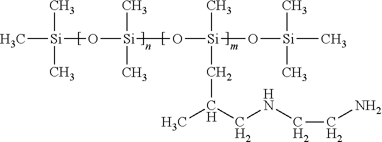

in which m and n are numbers, the sum of which (m+n) is between about 1 and about 2000, preferably between about 50 and about 150, and n preferably has values from about 0 to about 1999 and especially from about 49 to about 149 and m preferably having values from about 1 to about 2000 and especially from about 1 to about 10.

In a further, preferred embodiment, cosmetic preparations contain an aminofunctional silicone of formula (Si-1b)

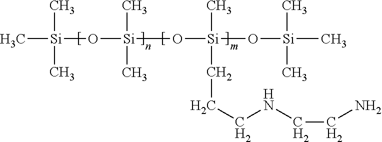

in which m and n are numbers, the sum of which (m+n) is between about 1 and about 2000, preferably between about 50 and about 150, and n preferably has values from about 0 to about 1999 and especially from 49 to 149, m preferably having values from about 1 to about 2000 and especially from about 1 to about 10. These silicones of formula (Si1-a) and (Si1-b) are referred to as amodimethicones according to the INCI declaration. Corresponding amodimethicones are obtainable, for example, in the form of an emulsion as a commercial product, Dow Corning 939 or as a commercial product, DOW Corning 949.

In an alternative embodiment, the aminofunctional silicone is a functionalized amodimethicone. Corresponding functionalized amodimethicones are described, for example, by the formula (Si-1c)

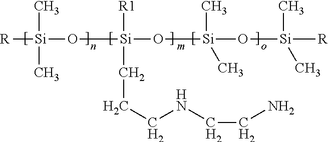

wherein
R represents —OH. an (optionally ethoxylated and/or propoxylated) (C₁ to C₂₀) alkoxy group or a CH₃ group,
R1 represents —OH, a (C₁ to C₂₀) alkoxy group or a —CH₃ group and
m, n₁ and n₂ are numbers, the sum of which (m+n1+n2) is between about 1 and about 2000, preferably between about 50 and about 150, and (n1+n2) preferably assumes values from about 0 to about 1999 and especially from about 49 to about 149, m preferably having values from about 1 to about 2000 and especially from about 1 to about 10.

Amino functional silicone a2) from the group of hydroxylamino-modified silicones, for example, in which, in the above formula (Si-2b), R represents OH, are preferred. Corresponding silicones with the INCI name of amodimethicones are obtainable as a commercial product, Wacker Belsil ADM 652.

If the R group represents (an optionally ethoxylated and/or propoxylated) (C₁ to C₂₀) alkoxy group, then, for example, (C₁₃ to C₁₅ alkoxy) PG amodimethicone (for example, as a commercial product) is included with this general formula (S1-2b): DC 8500 from Dow Corning), Trideceth-9 PG-amodimethicone (obtainable, for example as a commercial product, Silcare Silicone SEA from the Clariant company).

Regardless of the aminofunctional silicones, which are used, inventive cosmetic preparations are preferred, which contain an amino functional silicone, the amine number of which is greater than 0.25 meq/g, preferably greater than 0.3 meq/g and particularly greater than 0.4 meq/g. The amine number represents the milliequivalents of amine per gram of the aminofunctional silicone. It can be determined by titration and also stated in the unit of mg KOH/g.

Preferred cosmetic compositions are exemplified in that, based on their total weight, these compositions furthermore contain from about 0.01 to about 2.0% by weight, preferably from about 0.05 to about 1.0% by weight and particularly from about 0.1 to about 0.8% by weight of silicone, preferably in the form of an amino functional silicone.

The nonionic surfactants form a second group of preferably used optional active and auxiliary materials. The nonionic surfactants constitute preferably from about 0.05 to about 2.0% by weight, especially from about 0.1 to about 1.0% by weight and particularly from about 0.1 to about 0.5% by weight of the total weight of the cosmetic preparation.

Alkylene oxide addition products of saturated, linear, fatty alcohols, fatty esters and fatty acids, each with from about 2 to about 80 moles of ethylene oxide and/or from about 1 to about 5 mole of propylene oxide per mole of fatty alcohol or fatty acid have proven to be preferred non-ionic surfactants. Preparations with excellent properties are also obtained if they contain fatty esters of ethoxylated glycerin as nonionic surfactants. In this connection, it has proven to be particularly advantageous if the further non-ionic surfactant has an HLB value above 10 and preferably above 14. For this, it is necessary that the non-ionic surfactant has a sufficiently high degree of ethoxylation. A further embodiment of the first object as contemplated herein therefore is exemplified in that the cosmetic preparation a) contains at least one ethoxylated surfactant having at least 30 ethylene oxide units as nonionic surfactant.

Cosmetic compositions which, based on their total weight, comprise from about 0.05 to about 2.0% by weight, preferably from about 0.1 to about 1.0% by weight and in particular from about 0.1 to about 0.5% by weight of at least one nonionic surfactant of the group of alkoxylated fatty alcohols, preferably at least one nonionic surfactant from the group of ethoxylated fatty alcohols is particularly preferred because of its stability and cosmetic properties.

In addition to the corresponding ethoxylated fatty alcohols, especially the addition products of from about 30 to about 60 moles of ethylene oxide and castor oil and hydrogenated castor oil are particularly suitable. Examples of such suitable surfactants have the INCI names of Steareth-30, Ceteareth-30, Oleth-30, Ceteareth-50, or PEG-40 hydrogenated castor oil and PEG-60 hydrogenated castor oil.

In summary, preferred compositions therefore are exemplified in that they comprise from about 0.05 to about 2.0% by weight, preferably from about 0.1 to about 1.0% by weight, and in particular from about 0.1 to about 0.5% by weight of at least one nonionic surfactant from the group comprising the addition products of from about 30 to about 60 moles of ethylene oxide and castor oil and hydrogenated castor oil, preferably at least one nonionic surfactant from the group comprising compounds with the INCI names Steareth-30, Ceteareth-30, Oleth-30, Ceteareth-50, PEG-40 hydrogenated castor oil and PEG-60 hydrogenated castor oil, especially PEG-40 hydrogenated castor oil.

Particularly preferred compositions are therefore exemplified in that these contain from about 0.05 to about 2.0% by weight, preferably from about 0.1 to about 1.0% by weight, and in particular from about 0.1 to about 0.5% by weight, of at least one of the addition products of from about 30 to about 60 moles of ethylene oxide and castor oil or hydrogenated castor oil.

To further optimize the product properties, in particular, to increase product stability and to improve the hair-care properties (handle, combability, elasticity, softness), it has proven to be advantageous if embodiments of the embodiments of the cosmetic compositions also contain at least one cationic surfactant.

Cationic surfactants of the quaternary ammonium compounds, the esterquats and the amidoamines types can be used as contemplated herein. Preferred quaternary ammonium compounds are ammonium halides, in particular chlorides and bromides, such as alkyltrimethylammonium chlorides, dialkyldimethylammonium chlorides and trialkylmethylammonium chlorides. The long alkyl chains of these surfactants preferably have 10 to 18 carbon atoms, such as, for example, in cetyltrimethylammonium chloride, stearyltrimethylammonium chloride, di-stearylimethylammonium chloride, Lauroyl-dimethylammonium chloride, lauryldimethylbenzylammonium chloride and tricetylmethylammonium chloride. Further preferred cationic surfactants are the imidazolium compounds known under the INCI names Quaternium-27 and Quaternium-83.

The use of alkyltrimethylammonium salts, particularly of C₁₂-C₂₀ alkyltrimethylammonium salts and particularly of C₁₆-C₁₈ alkyltrimethylammonium chlorides is preferred.

A further preferred embodiment as contemplated herein is exemplified in that the cosmetic composition additionally contains from about 0.05 to about 5.00% by weight, particularly preferably from about 0.1 to about 4.00% by weight and in particular from about 0.2 to about 3.00% by weight of at least one cationic surfactant, the quantities being based on the total weight of the cosmetic composition.

Particular preference is given to the use of C₁₂-C₂₀ alkyltrimethylammonium salts and, in particular, to the use of C₁₆-C₁₈ alkyltrimethylammonium chlorides in embodiments of the embodiments of the compositions in the abovementioned amounts.

A particularly preferred cosmetic composition for the temporary shaping of keratin-containing fibers, especially of human hair, containing in a cosmetic carrier contains, based upon the total weight of the latter,

a) from about 0.05 to about 2.0% by weight of chitosan or a chitosan derivative
b) from about 0.05 to about 2.0% by weight of a C₁-C₄ carboxylic acid
c) from about 0.05 to about 4.0% by weight of a vinylpyrrolidone homopolymer
d) from about 0.01 to about 2.0% by weight of amino functional silicone
e) from about 0.05 to about 2.0% by weight of the addition product of from about 30 to about 60 moles of ethylene oxide and castor oil or hydrogenated castor oil
f) from about 0.05 to about 5.0% by weight of cationic surfactant, wherein the cosmetic composition

is free of vinylpyrrolidone/vinyl acetate copolymer and cationic cellulose compounds, which result from the reaction of hydroxyethylcellulose with a dimethyldiallylammonium reactant, especially with dimethyldiallylammonium chloride or

is free of vinyl acetate/vinylpyrrolidone copolymer and N-methylvinylimidazole/vinylpyrrolidone copolymer.

A furthermore preferred embodiment as contemplated herein is exemplified in that the cosmetic composition, based on the total weight thereof, contains from about 1 to about 15%, especially from about 2 to about 12.5% and particularly from about 3 to about 10% of at least one propellant, preferably of propane and/or butane.

Embodiments of the Embodiments of the cosmetic compositions may contain further auxiliary care materials and additives. As care material, the cosmetic composition may contain, for example, at least one protein hydrolysate and/or one of its derivatives, Protein hydrolysates are product mixtures, which are obtained by the acidically, basically or enzymatically catalyzed degradation of proteins. The concept of protein hydrolysates as contemplated herein is understood to include also total hydrolysates, as well as individual amino acids and their derivatives and mixtures of different amino acids. The molecular weight of the protein hydrolysates, which can be used as contemplated herein, is between about 75, the molecular weight of glycine, and about 200,000; preferably it is between about 75 and about 50,000 and, particularly, between about 75 and about 20,000 Dalton.

As a care material, embodiments of the embodiments of the composition may furthermore contain at least one vitamin, one provitamin, one vitamin precursor and/or one of their derivatives. Moreover, as contemplated herein, those vitamins, pro vitamins and vitamin precursors are preferred, which usually are assigned to the groups A, B, C, E, F and H. Especially preferred are the vitamins of the groups A, B and E, especially niacinamide, panthenol and/or biotin.

As care material, embodiments of the materials may furthermore contain at least one plant extract, but also monosaccharides or oligosaccharides and/or lipids. As care materials, embodiments of the cosmetic compositions may also contain UV filters, which usually are used in amounts of from about 0.01 to about 1% by weight, based on the total weight of embodiments of the composition.

Embodiments of the cosmetic compositions preferably are free of hydrophobic, pyrogenic silica. For example, silicas, the surface of which is modified with hydrophobic groups such as alkyl groups, are referred to as hydrophobic, pyrogenic silicas. Such alkyl-modified, hydrophobic, pyrogenic silicas are commercially available, for example, under the trade name of Aerosil® R from Evonik Degussa, or under the trade name of Cab-O-Sil® TS from Cabot.

Embodiments of the cosmetic compositions contain their active ingredients in a cosmetic carrier, preferably in an aqueous cosmetic carrier, an alcoholic cosmetic carrier or an aqueous-alcoholic cosmetic carrier. For the purpose of temporarily shaping hair, such carriers are, for example, lotions, water-in-oil emulsions, oil-in-water emulsions, creams, gels, foams, pomades, waxes or other preparations, which are suitable for use on hair. As contemplated herein, the carrier preferably is a water-containing or an aqueous-alcoholic cosmetic carrier. Furthermore, as contemplated herein, it is preferred if the cosmetic carrier of embodiments of the composition contains water, so that the composition contains at least 50% by weight of water, based on the total weight of the total composition.

For the purposes of the present disclosure, aqueous-alcoholic carriers are understood to be aqueous compositions, which contain from about 3 to about 70% by weight of a C₁-C₄ alcohol, in particular, ethanol or isopropanol. Embodiments of the compositions may additionally contain further organic solvents, such as methoxy butanol, benzyl alcohol, ethyl diglycol, 1,2-propylene glycol or 1,3-propylene glycol. All water-soluble organic solvents are preferred.

A furthermore preferred embodiment as contemplated herein is exemplified in that the cosmetic composition, based on the total weight thereof, contains from about 50 to about 95%, especially from about 60 to about 92.5% and particularly from about 65 to about 90% of water or a water-alcohol mixture.

The compositions of some preferred cosmetic preparations, which

are free of vinylpyrrolidone/vinyl acetate copolymer and cationic cellulose compounds, which result from the reaction of hydroxyethylcellulose with a dimethyldiallylammonium reactant, especially with dimethyldiallylammonium chloride or

are free of vinyl acetate/vinylpyrrolidone copolymer and N-methylvinylimidazole/vinylpyrrolidone copolymer,

are given in the following Tables (in percent by weight of the total composition unless stated otherwise), The cosmetic compositions, listed in these Tables, preferably are free of cationic cellulose compounds, which result from the reaction of hydroxyethylcellulose and a dimethyldiallylammonium reactant, in particular dimethyldiallylammonium chloride, and which bear the INCI name polyquaternium-4. Preferably, the compositions are free of hydrophobic, pyrogenic silica.

| | Formula 1 | Formula 2 | Formula 3 | Formula 4 |
|---|---|---|---|---|
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Water | 50 to 95 | 60 to 92.5 | 60 to 92.5 | 70 to 90 |
| further constituents | ad 100 | ad 100 | ad 100 | ad 100 |
| | Formula 5 | Formula 6 | Formula 7 | Formula 8 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Water | 50 to 95 | 60 to 92.5 | 60 to 92.5 | 70 to 90 |
| further constituents | ad 100 | ad 100 | ad 100 | ad 100 |
| | Formula 9 | Formula 10 | Formula 11 | Formula 12 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| Water | 50 to 95 | 60 to 92.5 | 60 to 92.5 | 70 to 90 |
| further constituents | ad 100 | ad 100 | ad 100 | ad 100 |
| | Formula 13 | Formula 14 | Formula 15 | Formula 16 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| further constituents | ad 100 | ad 100 | ad 100 | ad 100 |
| | Formula 17 | Formula 18 | Formula 19 | Formula 20 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 21 | Formula 22 | Formula 23 | Formula 24 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| Water | 50 to 95 | 60 to 92.5 | 60 to 92.5 | 70 to 90 |
| further constituents | ad 100 | ad 100 | ad 100 | ad 100 |
| | Formula 25 | Formula 26 | Formula 27 | Formula 28 |
| Chitosan | 0.05 to 2.0 | 0.1 to 1.5 | 0.1 to 1.5 | 0.2 to 1.0 |
| Lactic | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| further constituents | ad 100 | ad 100 | ad 100 | ad 100 |
| | Formula 29 | Formula 30 | Formula 31 | Formula 32 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 33 | Formula 34 | Formula 35 | Formula 36 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| further constituents | ad 100 | ad 100 | ad 100 | ad 100 |
| | Formula 37 | Formula 38 | Formula 39 | Formula 40 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Aminofunctioneal silicones | 0.01 to 2.0 | 0.05 to 1.0 | 0.05 to 1.0 | 0.1 to 0.8 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 41 | Formula 42 | Formula 43 | Formula 44 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant. preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 45 | Formula 46 | Formula 47 | Formula 48 |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant. preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 49 | Formula 50 | Formula 51 | Formula 52 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant. preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| further constituents | ad 100 | ad 100 | ad 100 | ad 100 |
| | Formula 53 | Formula 54 | Formula 55 | Formula 56 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 57 | Formula 58 | Formula 59 | Formula 60 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor Oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 61 | Formula 62 | Formula 63 | Formula 64 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |
| | Formula 65 | Formula 66 | Formula 67 | Formula 68 |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Amodimethicone | 0.01 to 2.0 | 0.05 to 1.0 | 0.05 to 1.0 | 0.1 to 0.8 |
| PEG-40 hydrogenated castor oil | 0.05 to 2.0 | 0.1 to 1.0 | 0.1 to 1.0 | 0.1 to 0.5 |
| Cetrimonium chloride | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| further constituents | ad 100 | ad 100 | ad 100 | ad 100 |

According to a further preferred embodiment, embodiments of the cosmetic compositions are present as a foam. For this purpose, inventive the compositions are packaged in a dispensing device, which is either a compressed gas container (aerosol container) additionally filled with a propellant or a nonaerosol container. The compressed gas containers, with the aid of which a product is distributed by the internal gas pressure of the container via a valve, are referred to as an “aerosol container”. A container under normal pressure, with the help of which a product is distributed by employing mechanical action by a pump or squeezing system, is defined conversely to the aerosol definition as a “non-aerosol” container.

Particularly preferred within this embodiment, embodiments of the cosmetic compositions are present as an aerosol foam in an aerosol container. embodiments of the composition therefore preferably contains additionally at least one propellant. Inventive compositions, which are present in the form of an aerosol product, can be produced in the usual manner. As a rule, all the constituents of embodiments of the composition, with the exception of the propellant, are filled into a suitable pressure-resistant container, which is then closed with a valve. The desired quantity of propellant is finally introduced by conventional techniques.

In the embodiment as an aerosol foam, inventive propellants are, for example, selected from N₂O, dimethyl ether, CO₂, air, alkanes having from about 3 to about 5 carbon atoms, such as propane, n-butane, isobutane, n-pentane and isopentane and the mixtures thereof. According to the embodiment of an aerosol foam, the abovementioned alkanes, mixtures of the alkanes mentioned or mixtures of the alkanes mentioned with dimethyl ether are used as the only blowing agent. However, the invention explicitly also includes the use of propellants of the type of the fluorochlorohydrocarbons, especially however the fluorohydrocarbon. Dimethyl ether, propane, n-butane, iso-butane and mixtures thereof are particularly preferred. Especially preferred are mixtures of propane and butane, which are used as the sole propellant in the ratio by weight of propane to butane of from about 70 to about 30 to about 15 to about 85. As contemplated herein, butane is understood to be n-butane, isobutane and mixtures of n-butane and isobutane.

The propellants preferably are used in embodiments of the cosmetic compositions in an amount of from about 1 to about 15% by weight, particularly preferably of from about 2 to about 12.5% by weight, and in particular of from about 3 to about 10% by weight, based on the weight of the total composition, propane, butane and propane/butane mixtures being preferred.

The compositions of some preferred cosmetic preparations, which
are free of vinylpyrrolidone/vinyl acetate copolymer and cationic cellulose compounds, which result from the reaction of hydroxyethylcellulose with a dimethyldiallylammonium reactant, especially with dimethyldiallylammonium chloride, or
are free of vinyl acetate/vinylpyrrolidone copolymer and N-methylvinylimidazole/vinylpyrrolidone copolymer
   are given in the following Tables (in percent by weight of the total composition unless stated otherwise). The cosmetic compositions, listed in these Tables, preferably are free of cationic cellulose compounds, which result from the reaction of hydroxyethylcellulose and a dimethyldiallylammonium reactant, in particular dimethyldiallylammonium chloride, and which bear the INCI name Polyquaternium-4. Preferably, the compositions are free of hydrophobic, pyogenic silica.

| | Formula 1a | Formula 2a | Formula 3a | Formula 4a |
|---|---|---|---|---|
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Water | 50 to 95 | 60 to 92.5 | 60 to 92.5 | 70 to 90 |
| Propellant. especially | 1.0 to 15 | 2.0 to 12.5 | 2.0 to 12.5 | 3.0 to 10 |
| propane/butane | | | | |
| further constituents | ad 100 | ad 100 | ad 100 | ad 100 |
| | Formula 5a | Formula 6a | Formula 7a | Formula 8a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 9a | Formula 10a | Formula 11a | Formula 12a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 13a | Formula 14a | Formula 15a | Formula 16a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 17a | Formula 18a | Formula 19a | Formula 20a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 21a | Formula 22a | Formula 23a | Formula 24a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 25a | Formula 26a | Formula 27a | Formula 28a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 29a | Formula 30a | Formula 31a | Formula 32a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 33a | Formula 34a | Formula 35a | Formula 36a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Propellant, especially propane/ | 1.0 to 15 | 2.0 to 12.5 | 2.0 to 12.5 | 3.0 to 10 |
| butane | | | | |
| Water | 50 to 95 | 60 to 92.5 | 60 to 92.5 | 70 to 90 |
| further constituents | ad 100 | ad 100 | ad 100 | ad 100 |
| | Formula 37a | Formula 38a | Formula 39a | Formula 40a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 41a | Formula 42a | Formula 43a | Formula 44a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 45a | Formula 46a | Formula 47a | Formula 48a |
| Chitosan | 0.05 to 2.0 | 0.1 to 1.5 | 0.1 to 1.5 | 0.2 to 1.0 |
| C₁-C₄ Carboxylic acid | 0.05 to 2.0 | 0.05 to 2.0 | 0.1 to 1.5 | 0.2 to 1.0 |
| vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 49a | Formula 50a | Formula 51a | Formula 52a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 53a | Formula 54a | Formula 55a | Formula 56a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 57a | Formula 58a | Formula 59a | Formula 60a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| Vinylpyrrolidone homopolymer | 0.05 to 4.0 | 0.3 to 3.0 | 0.3 to 3.0 | 0.5 to 2.5 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 61a | Formula 62a | Formula 63a | Formula 64a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| | 0.05 to 4.0 | 0.3 to 3.0 | 0.3 to 3.0 | 0.5 to 2.5 |
| aminofunctional silicone | 0.01 to 2.0 | 0.05 to 1.0 | 0.05 to 1.0 | 0.1 to 0.8 |
| nonionic surfactant, preferably | 0.05 to 2.0 | 0.1 to 1.0 | 0.1 to 1.0 | 0.1 to 0.5 |
| PEG-40 hydrogenated castor oil | | | | |
| cationic surfactant | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propellant, especially propane/ | 1.0 to 15 | 2.0 to 12.5 | 2.0 to 12.5 | 3.0 to 10 |
| butane | | | | |
| further constituents | ad 100 | ad 100 | ad 100 | ad 100 |
| | Formula 65a | Formula 66a | Formula 67a | Formula 68a |
| Chitosan | 0.05 to 2.0 | 0.1 to 1.5 | 0.1 to 1.5 | 0.2 to 1.0 |
| Lactic acid | 0.05 to 2.0 | 0.05 to 2.0 | 0.1 to 1.5 | 0.2 to 1.0 |
| vinylpyrrolidone homopolymer | 0.05 to 4.0 | 0.3 to 3.0 | 0.3 to 3.0 | 0.5 to 2.5 |
| Amodimethicone | 0.01 to 2.0 | 0.05 to 1.0 | 0.05 to 1.0 | 0.1 to 0.8 |
| PEG-40 hydrogenated castor oil | 0.05 to 2.0 | 0.1 to 1.0 | 0.1 to 1.0 | 0.1 to 0.5 |
| Cetrimonium chloride | 0.05 to 5.0 | 0.1 to 4.0 | 0.1 to 4.0 | 0.2 to 3.0 |
| Water | 50 to 95 | 60 to 92.5 | 60 to 92.5 | 70 to 90 |
| Propane/butane | 1.0 to 15 | 2.0 to 12.5 | 2.0 to 12.5 | 3.0 to 10 |
| further constituents | ad 100 | ad 100 | ad 100 | ad 100 |

As stated at the beginning, embodiments of the cosmetic compositions are suitable for the temporary shaping of keratinic fibers and for improving the curl retention, especially the high humidity curl retention. The corresponding uses of embodiments of the composition are therefore further objects of this application.

The last application objects are:

A method for the temporary shaping of keratinic fibers, especially of human hair, for which the cosmetic composition of one of the preceding points is applied on keratinic fibers and

a method for improving the curl retention, especially the high humidity curl retention of keratinic fibers, especially human hair, for which an inventive cosmetic composition is applied on keratinic fibers, especially human hair.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for the temporary shaping of keratin-containing fibers, comprising in a cosmetic carrier, based upon the total weight of the cosmetic composition,
a) from about 0.05 to about 2.0% by weight of chitosan or a chitosan derivative
b) from about 0.05 to about 2.0% by weight of a C₁-C₄ carboxylic acid
c) from about 0.05 to about 4.0% by weight of a vinylpyrrolidone homopolymer wherein the cosmetic composition
is free of vinyl acetate/vinylpyrrolidone copolymer and cationic cellulose compounds, which result from the reaction of hydroxyethylcellulose with a dimethyldiallylammonium reactant, or
is free of vinyl acetate/vinylpyrrolidone copolymer and N-methylvinylimidazole/vinylpyrrolidone copolymer.

2. The cosmetic composition of claim 1, wherein the percentage by weight of the chitosan or chitosan derivative, in the total weight of the cosmetic composition, is from about 0.1 to about 1.5% by weight.

3. The cosmetic composition of claim 1, wherein the percentage by weight of the C₁-C₄ carboxylic acid in the total weight of the cosmetic composition, is from about 0.1 to about 1.5% by weight.

4. The cosmetic composition of claim 1, wherein the C₁-C₄ carboxylic acid is selected from the group comprising formic acid and lactic acid.

5. The cosmetic composition of claim 1, wherein the percentage by weight of the vinylpyrrolidone homopolymer in the total weight of the cosmetic composition, is from about 0.3 to about 3.0% by weight.

6. The cosmetic composition of claim 1, wherein the composition, based on the total weight thereof, furthermore comprises
e) from about 0.05 to about 2.0% by weight of at least one nonionic surfactant from the group comprising the addition products of 30 to 60 moles of ethylene oxide and castor oil and hydrogenated castor oil.

7. The cosmetic composition of claim 1, wherein the cosmetic composition, based on the total weight thereof, comprises from about 0.05 to about 5.0% by weight of at least one cationic surfactant.

8. The cosmetic composition of claim 1, wherein the cosmetic composition is utilized for the temporary shaping of keratinic fibers.

9. The cosmetic composition of claim 1, wherein the cosmetic composition is utilized for improving the curl retention.

10. A method for the temporary shaping of keratinic fibers for which the cosmetic composition of claim 1 is applied on keratinic fibers.

11. The cosmetic composition of claim 1, wherein the dimethyldiallylammonium reactant comprises dimethyldiallylammonium chloride.

12. The cosmetic composition of claim 6, wherein the at least one nonionic surfactant is from the group comprising compounds with the INCI names Steareth-30, Ceteareth-30, Oleth-30, Ceteareth-50, PEG-40 hydrogenated castor oil and PEG-60 hydrogenated castor oil.

13. The cosmetic composition of claim 12, wherein the at least one nonionic surfactant comprises PEG-40 hydrogenated castor oil.

14. The cosmetic composition of claim 1, wherein the cosmetic composition, based on the total weight thereof, comprises from about 50 to about 95% of water or of a mixture of water and alcohol.

15. The cosmetic composition of claim 1, wherein the cosmetic composition, based on the total weight thereof, further comprises d) from about 0.01 to about 2.0% by weight of silicone.

16. The cosmetic composition of claim 1, wherein the cosmetic composition, based on the total weight thereof, further comprises f) from about 0.05 to about 5.0% by weight of at least one cationic surfactant.

17. The cosmetic composition of claim 1, wherein the cosmetic composition, based on the total weight thereof, comprises from about 1 to about 15% of propane and/or butane.

18. The cosmetic composition of claim 1, wherein the cosmetic composition is free of hydrophobic, pyrogenic silica.

19. The cosmetic composition of claim 1, wherein the cosmetic composition is free of vinyl acetate/vinylpyrrolidone copolymer and cationic cellulose compounds, which result from the reaction of hydroxyethylcellulose with a dimethyldiallylammonium reactant.

20. The cosmetic composition of claim 1, wherein the cosmetic composition is free of vinyl acetate/vinylpyrrolidone copolymer and N-methylvinylimidazole/vinylpyrrolidone copolymer.
